(19) United States
(12) Patent Application Publication
(10) Pub. No.: US 2019/0060593 A1
(43) Pub. Date: Feb. 28, 2019

(54) MEDICO-SURGICAL TUBES AND THEIR MANUFACTURE

(71) Applicant: SMITHS MEDICAL INTERNATIONAL LIMITED, Ashford (GB)

(72) Inventor: Eric Pagan, Hythe (GB)

(73) Assignee: SMITHS MEDICAL INTERNATIONAL LIMITED, Ashford (GB)

(21) Appl. No.: 15/767,287

(22) PCT Filed: Sep. 28, 2016
(86) PCT No.: PCT/GB2016/000169

§ 371 Date: Apr. 10, 2018

(30) Foreign Application Priority Data
Oct. 24, 2015 (GB) 1518888.1

(51) Int. Cl. A61M 16/04; A61L 29/04

(52) U.S. Cl. CPC A61M 16/0427; A61L 29/041; A61M 16/0434; A61M 2207/00; A61M 16/0465

(57) ABSTRACT

A tracheostomy tube assembly includes an outer tracheostomy tube (1) and a removable inner cannula (3). The inner cannula (3) is made by helically winding PTFE tape (40) on a mandrel (42) and applying a reinforcing strip (34) of PTFE or other materials longitudinally along the outside of the wound tape. The tape and strip are then heated to sinter and bond them together and convert the PTFE to ePTFE and form an inner layer. An outer layer (46) is extruded along the outside of the inner layer. The shaft (30) is cut between the ends of adjacent reinforcing strips (34) to leave a short unreinforced portion (35) at the patient end (32) of each shaft.

This invention relates to medico-surgical tubes and to methods of manufacture of such tubes.

Tracheostomy tube assemblies commonly include an outer tube and an inner tube or cannula that is a removable fit within the outer tube. The inner tube can be removed and replaced periodically to ensure that the passage through the assembly does not become blocked by secretions. This avoids the need frequently to remove the outer tube.

The inner tube presents various problems because it must be thin walled and a close fit within the outer tube so as to limit the resistance to flow of gas along the assembly. It must, however, also be sufficiently stiff to be inserted in the outer tube without buckling or kinking. A particularly suitable material for the inner cannula is PTFE or expanded PTFE (ePTFE). The use of such a material in an inner cannula is described in WO94/01156 and in WO2004/101048. The Flextra tube previously sold by Tyco Healthcare was made of ePTFE. U.S. Pat. No. 8,419,075 describes an inner cannula of ePTFE attached with a hub at one end by an overmoulding technique. Whilst such a material has various advantages it also has a problem of poor axial stability in that it can be compressed axially by a relatively small axial force. This is a problem because, if the inner cannula cannot be freely inserted in the outer tube, such as because of a deposit or other obstruction on the inside of the outer tube, the inner cannula could be partly compressed and restrict gas flow along the assembly. It has also been found that the action of cleaning the cannula with a brush or swab inserted along its length can change the dimensions of the cannula. It is difficult to strengthen an inner cannula of ePTFE because this material does not bond well to other materials.

It is an object of the present invention to provide an alternative tube and a method of its manufacture.

According to one aspect of the present invention there is provided a method of making a medico-surgical tube including the steps of helically winding a tape of PTFE about a mandrel to form a shaft, applying lengths of separate reinforcing strips of plastics material along the shaft and spaced longitudinally from one another, sintering the shaft before or after the lengths of reinforcing strips are applied so that edges of the tape are bonded with one another and the PTFE is converted to ePTFE, and subsequently cutting the length of sintered wound tape into separate shafts at locations between the strips so that each shaft has an end portion without a reinforcing strip that is more flexible than the remainder of the shaft.

The reinforcing strips may be of PTFE. The strips may have formations along their length to enable the strips to bend more readily in the plane of the strip. The method may include the step of applying a plurality of strips aligned parallel with one another. The method may include the step of applying an outer layer around the wound tape and the strips to encase the strips and the tape. The outer layer may be provided by an extruded tubular sleeve. The method may include the step of applying a visible marking in alignment with the strips. The method may include the step of attaching a hub to the machine end of the shaft. The hub may be overmoulded onto the machine end of the shaft.

According to another aspect of the present invention there is provided a medico-surgical tube made by a method according to the above one aspect of the present invention.

According, to a further aspect of the present invention there is provided a medico-surgical tube, characterised in that the tube includes an inner layer formed by a helical PTFE tape with heat-bonded edges, one or more reinforcing strips extending axially along the outside of the inner layer and an outer layer covering the inner layer and the or each reinforcing strips and that and that the tube has a portion at its patient end unreinforced by the or each reinforcing strip.

According to yet another aspect of the present invention there is provided an assembly of an outer tracheostomy tube and an inner tube according to the above other or further aspect of the present invention or made by a method according to the above one aspect of the present invention.

A tracheostomy assembly with an inner cannula and its method of manufacture according to the present invention will now be described, by way of example, with reference to the accompanying drawings, in which:

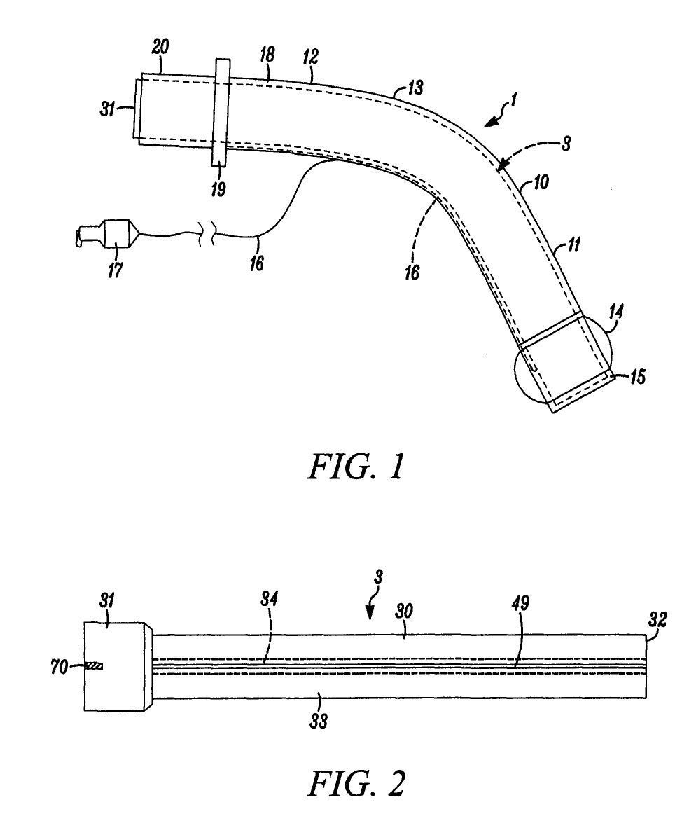

FIG. 1 shows the assembly schematically;

FIG. 2 is a plan view of the inner cannula;

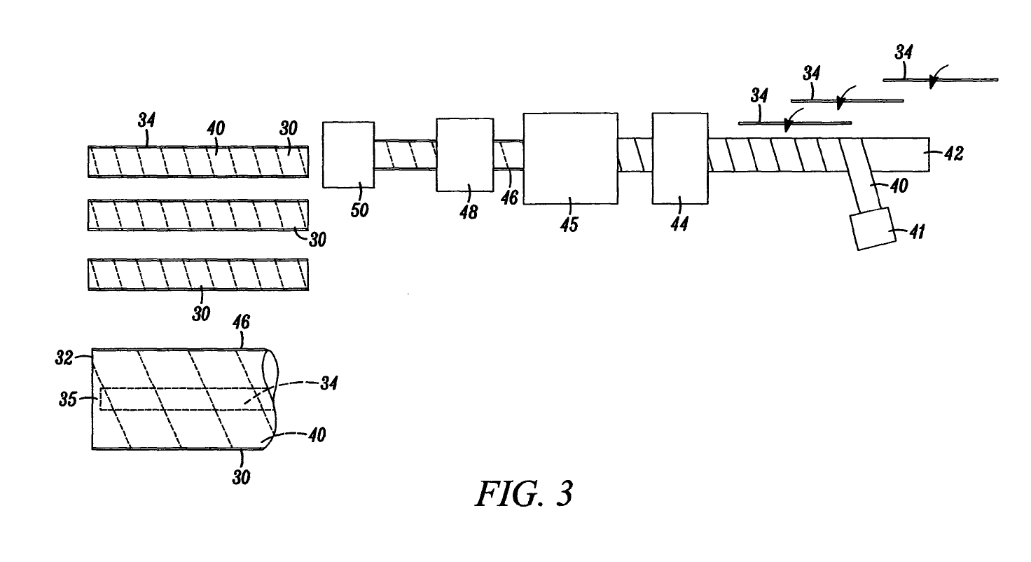

FIG. 3 illustrates initial steps in the method of making the inner cannula;

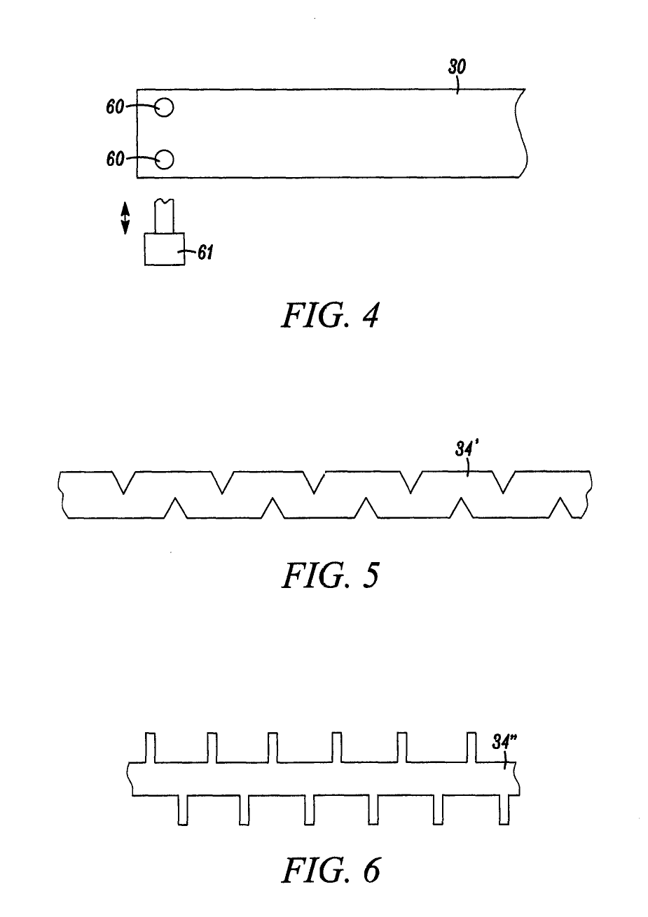

FIG. 4 is a side elevation view showing a subsequent step in the method of making the cannula;

FIG. 5 is a plan view of an alternative reinforcing strip; and

FIG. 6 is a plan view of a further alternative reinforcing strip.

With reference first to FIG. 1, the tracheostomy tube assembly comprises an outer tube 1 and an inner tube or cannula 3, which is removable from the outer tube so that it can be periodically replaced in the usual way.

The outer tube 1 is entirely conventional having a shaft 10 with straight forward or patient end section 11 and rear or machine end section 12 joined by a curved section 13. Alternative outer tubes could be smoothly curved along their entire length or could be highly flexible and reinforced with a natural straight shape. A sealing cuff 14 embraces the shaft 10 close to its patient end 15. The cuff 14 can be inflated for sealing, or deflated for insertion and removal, via an inflation line 16 and a combined inflation indicator balloon and coupling 17. At its rear end 18, the outer tube 1 has a flange 19 to which a tape (not shown) can be attached for securing the assembly around the neck of the patient. A hub 20 projects from the machine side of the flange 19 by which gas connection can be made to the tube 1. In use, the tube 1 extends through a surgically-made tracheostomy opening in the neck, with the patient end 15 of the tube 1 located in the trachea. The cuff 14 is inflated to form a seal between the outside of the tube and the tracheal wall so that gas flow is confined along the bore of the tube. The machine end 18 of the tube 1 extends externally of the tracheostomy.

With reference now also to FIG. 2, the inner tube or cannula 3 comprises a shaft 30 and a hub or machine end fitting 31. The inner cannula 3 is about 194 mm long and its shaft 30 has an internal diameter of about 8 mm with an external diameter of about 9 mm along the major part of its length. Other sizes of inner cannula could be provided. In use, the cannula 3 extends as a close sliding fit within the bore of the outer tube 1 with the patient end 32 of the cannula extending substantially level with the patient end 15 of the outer tube and with its machine end fitting 31 locating in the hub 20 of the outer tube.

The shaft 30 comprises a wall 33 of ePTFE and a strengthening member in the form of a reinforcing strip 34 extending longitudinally along its length. The reinforcing strip 34 may also be of ePTFE or it may be of a material different from and stiffer than that of the wall 33 such as polyethylene, polypropylene, PVC, nylon, polyester, EVA or polyurethane. The strip 34 increases the axial stiffness of the shaft 30, reducing the risk that the shaft would be axially compressed by any axial force applied during normal use. The strip 34 still enables the shaft 30 to be bent although it gives the shaft a plane of preferential bending that includes the strip. To insert the inner cannula 3 in the tube 1 the strip 34 is aligned with the outside of the curve of the tube so that the inner cannula can readily bend along the curve of the tube.

The inner cannula 3 is made in the manner shown in FIG. 3. The shaft 30 is formed in several steps shown from right to left in the drawing. First, a tape 40 of PTFE is wound from a reel 41 about a rotating mandrel 42 of circular section so that the adjacent turns of the helix contact one another. Alternatively, the PTFE tape 40 could be produced directly from an extrusion machine. The next step is for lengths of reinforcing strips 34 to be laid longitudinally one after the other along the outside of the helically-wound tape 40. The strips 34 are cut to a length that is about slightly shorter than the desired length of the cannula shafts 30 so that a short portion at the patient end of the cannula is unreinforced. Typically the strips 34 would be more than 0.5 mm shorter than the cannula shaft 34 but not greater than the diameter of the shaft (around 6 mm to 8 mm) so as to minimise any change in the overall length of the cannula caused during use. The strips 34 may, for example, be delivered by pulsed delivery or by a transfer carrier.

The next step takes place in a heater unit 44 through which the mandrel 42, wound tape 40 and strips 34 are passed. The heater 44 raises the temperature of the tape 40 sufficiently for it to be sintered and converted into ePTFE. At the same time, this causes the contacting adjacent edges of turns of the tape 40 to weld together to form a complete tube. Where the reinforcing strips 34 are also of PTFE these will similarly be converted to ePTFE and sintered to the underlying tape 40.

The next step is carried out by an extrusion station 45 where an outer layer in the form of a tubular sleeve 46 is extruded about the wound tape 40 and the reinforcing strips 34. Alternatively, the outer layer could be formed by spraying. The purpose of the outer sleeve 46 is to encase the tape 40 and reinforcing strip 34 to ensure a smooth outer surface to the finished shaft 30 and to retain the reinforcing strip securely in position, which is particularly important if the strip is of a material that is incompatible with that of the tape, that is, a material that does not bond readily with PTFE. Instead of a tubular outer layer it could be formed by applying a second wound tape around the outside of the inner helical tape and strips. The outer sleeve is typically about 0.1 mm thick so needs to have a uniform wall thickness to avoid the risk of perforations, making coating or co-extrusion the preferred technique and ePTFE the preferred material. The tubing emerging from the extrusion station 45 passes to a printing or marking unit 48 where the tubing is printed with visible line 49 (FIG. 2) on top of the reinforcing strip 43. The line 49 may be continuous or interrupted, or alternative markings could be provided, such as, for example, a short mark at a position that will be adjacent the machine end of the shaft. The purpose of the line or marking 49 is so that the position of the reinforcement line can be readily identified. The printing unit 48 could also print legends on the outside of the tubing, such as to indicate its internal diameter, manufacturer's name and the like. Alternatively, the reinforcing strip 43 could be of a different colour visible through a transparent or translucent outer sleeve. In another embodiment the outer sleeve could pre-printed with a stripe so as to avoid the need for the separate printing stage to mark the location of the embedded strip.

The next step is carried out by a cutting station 50 where the tubing is cut into lengths corresponding to that of the shaft 30. In particular, the tube is cut close to one end of the reinforcing strip 34 so that the opposite end of the cut lengths has a short reinforced portion 35 that is more flexible and provides the patient end of the cannula 3.

The cannula 3 is completed by attaching the hub 31 to the machine end of the shaft 30, the hub being of a relatively rigid material such as a flowable thermoplastic, for example, low density polyethylene. This material does not bond to PTFE or ePTFE but can be attached to the shaft 30 in a manner similar to that described in U.S. Pat. No. 8,419,075. As shown in FIG. 4, the machine end of the shaft 30 is formed with several openings 60 through its wall such as by a punch 61. Alternatively, openings could be formed by drilling, slitting, notching or in any other way. The material forming the hub 31 is then overmoulded over the machine end of the shaft 30 so that the material of the hub flows into these openings to form, when set, a mechanical anchor with the shaft. Alternatively, the hub could be formed on the shaft by insert moulding.

The hub 31 is formed with a mark 70 such as notch, projection or printed mark to indicate the location of the reinforcing strip 34. The reinforcing strip 34 gives the inner cannula 3 a preferential plane of bending including the length of the strip and the axis of the cannula. The hub 31 may be shaped to fit within the hub 20 on the outer tube 1 only in a particular orientation so that the reinforcing strip 34 lies in the plane of curvature of the outer tube.

The method of the present invention can be used to make an inner cannula or the like that is highly flexible yet also has sufficient axial strength to resist axial compression and extension forces during insertion into and removal from the outer tube and during cleaning by a swab or brush inserted into the cannula.

There are various ways in which the tube and method could be modified. For example, the cannula could include more than one reinforcing strip, such as, for example, two strips aligned parallel with and extending diametrically opposite one another. The or each strip 34′ may be patterned or notched, as shown in FIG. 5, to enable it to be bent more readily in the plane of the strip. The strip 34″ could have a central strip and two rows of ribs projecting laterally outwardly on opposite sides of the central strip as shown in FIG. 6.

The invention is not limited to tracheostomy tube inner cannulae and methods of making inner cannulae for tracheostomy tubes but could be used for other medico-surgical tubes and methods of making such tubes.

1-13. (canceled)

14. A method of making a medico-surgical tube including the steps of helically winding a tape of PTFE about a mandrel to form a shaft, applying lengths of separate reinforcing strips of plastics material along the shaft and spaced longitudinally from one another, sintering the shaft before or after the lengths of reinforcing strips are applied so that edges of the tape are bonded with one another and the PTFE is converted to ePTFE, and subsequently cutting the length of sintered wound tape into separate shafts at locations between the strips so that each shaft has an end portion without a reinforcing strip that is more flexible than the remainder of the shaft.

15. A method according to claim 14, characterised in that the reinforcing strips (34) are of PTFE.

16. A method according to claim 14, characterised in that the strips have formations along their length to enable the strips to bend more readily in the plane of the strips.

17. A method according to claim 14, characterised in that the method includes the step of applying a plurality of strips aligned parallel with one another.

18. A method according to claim 14, characterised in that the method includes the step of applying an outer layer around the wound tape and the strips to encase the strips and the tape.

19. A method according to claim 18, characterised in that the outer layer is provided by an extruded tubular sleeve.

20. A method according to claim 14, characterised in that the method includes the step of applying a visible marking in alignment with the strips.

21. A method according to claim 14, characterised in that the length of the reinforcing strips is such as to leave a short portion at the patient end of the cannula that is unreinforced.

22. A method according to claim 14, characterised in that the method includes the step of attaching a hub to the machine end of the shaft.

23. A method according to claim 22, characterised in that the hub is overmoulded onto the machine end of the shaft.

24. A medico-surgical tube, characterised in that the tube includes an inner layer formed by a helical PTFE tape with heat-bonded edges, one or more reinforcing strips extending axially along the outside of the inner layer and an outer layer covering the inner layer and at least one of the reinforcing strips and that the tube has a portion at its patient end unreinforced by the at least one reinforcing strip.

25. A tracheostomy tube assembly including an outer tracheostomy tube and an inner tube, characterised that the inner tube includes an inner layer formed by a helical PTFE tape with heat-bonded edges, one or more reinforcing strips extending axially along the outside of the inner layer and an outer layer covering the inner layer and at least one of the reinforcing strips and that the tube has a portion at its patient end unreinforced by the at least one reinforcing strip.

26. The tracheostomy tube assembly of claim 25, wherein the inner tube is made by a method that includes the steps of helically winding the PTFE tape about a mandrel to form a shaft, applying lengths of separate reinforcing strips along the shaft and spaced longitudinally from one another, sintering the shaft before or after the lengths of reinforcing strips are applied so that edges of the tape are bonded with one another and the PTFE is converted to ePTFE, and subsequently cutting the length of sintered wound tape at locations between the strips so that shaft has an end portion without a reinforcing strip that is more flexible than the remainder of the shaft.
